(19) United States
(12) Patent Application Publication
(10) Pub. No.: US 2007/0078467 A1
(43) Pub. Date: Apr. 5, 2007

(54) SURGICAL DEPILATORY DEVICE

(76) Inventor: Gary J. Mullen, Boston, MA (US)

Correspondence Address: BLACK LOWE & GRAHAM, PLLC, 701 FIFTH AVENUE, SUITE 4800, SEATTLE, WA, 98104, US

(21) Appl. No.: 11/531,648

(22) Filed: Sep. 13, 2006

Related U.S. Application Data
(60) Provisional application No. 60/716,567, filed on Sep. 13, 2005.

(51) Int. Cl. A61B 17/50

(52) U.S. Cl. 606/134

(57) ABSTRACT

A depilatory device includes flexible sheets that are coupled together to define a pouch. At least one side of the pouch is at least partially covered with an adhesive layer. Before or during a surgical operation, a user places their hand in a pocket formed in the pouch and then places the pouch in contact with a patient's skin to remove potential contaminants, such as hair, debris, and other particulates. The flexibility of the pouch permits it to readily conform to various contours of the patient. The pouch, after picking the aforementioned contaminants, can then be inverted (e.g., flipped inside-out) and sealed to keep the contaminants within the inverted pouch and not allow them back into the surgical environment. The inverted pouch can then be disposed of in a biological waste bin, for example.

### FIELD OF THE INVENTION

The present device generally relates to the removal and entrapment of hair and foreign debris in a sterile, surgical environment, and more specifically relates to the removal and entrapment of hair and debris without contaminating the sterile environment.

### BACKGROUND OF THE INVENTION

In a sterile environment, such as a hospital operating room, protocols exist to removal all contaminating or potentially contaminating hair, debris, and foreign substances that may exist on the skin of a patient or those in proximity to the patient. These protocols are directed at specifically diminishing the likelihood of peri-operative or post-operative infection of the patient through introduction of hair, debris, or foreign substances at the surgical incision, percutaneous, or wound site (e.g., surgical site). For example, such sterilization protocols in a hospital operating room may include the donning of surgical personnel garments, hair and face covers, sterile gowns, and sterile gloves. In addition, sterilization protocols typically include shaving and cleansing the surgical site to remove and extract hair and debris from around the anticipated surgical site.

These measures have their shortcomings. Shaving, for example includes the suspension of severed hairs in a soapy emulsion on the surface of the patient's skin, which when rinsed, is diluted and generally moved from the surgical incision site. But, the soapy emulsion may not be completely removed because of skin contours, crevices, limited time to prep the surgical site, or inadequate drying of the skin.

Additionally, razors are themselves intrusive instruments, having a sharp edge and operating by running that edge across the skin. Microscopic and larger cuts to the epidermis of the skin provide additional potential infection sites, and are well-configured to trap the soapy residue containing the emulsion and the suspended hair and debris promoting the culturing of infection at each of the razor-inflicted wound sites. Epidermal trauma is well-known to harbor bacteria and other pathogens by providing a microscopic nidus where the bacteria are less-effectively killed by the surgical skin scrubbing agent.

Shaving severs hair and loosens foreign debris that the hair and foreign debris are brushed aside by the action of the razor but not eliminated. The protocols generally in use simply move the hair away from the incision site, often to the floor of the surgical operatory, or alternatively pick the hair up by using adhesive tape to stick to the loose severed hair. Frequently however, the hair falls into the sheets covering the operating room table, crevices on the patient's body, and on the operating room floor. The multitude of other tasks that surgical personnel must perform prior to the actual operation (patient skin prep, attaching monitoring equipment to the patient, completion of the anesthesia induction, surgical scrubbing, instrument set-up, etc) usually prevents a great amount of time being spent retrieving all of the patient's shaved hair. Loose hair in the sterile environment is well known harbor pathogens, and increases the rate of post-operative wound infection. Lastly, loose hair on the floor increases the time necessary to clean the room for the next surgery, interrupts the surgical teams sure footing with the floor, and may by its movement in the surgical environment release further debris into the air that might arrive at the incision site in the course of surgery.

Some inventors have taught methods to suitably prevent the mechanisms for infection. U.S. Pat. No. 4,251,914 to Grosjean teaches a razor specifically designed to effectively shave a surgical site with minimal epidermal trauma. While suitably protecting the epidermis, the Grosjean razor does not collect the severed hair and loosened debris.

U.S. Pat. No. 4,282,877 to Mathews teaches the use of strips of sheet material of a pre-determined size and shape, which can be applied without heating to an area of body hair, and removed, thereby removing the hair. The strips use a combination of a high molecular weight polymer (as an example hydrogenated rosin), a tackifier (as an example styrene polymer), and a softening agent (as an example mineral oil) to entrap hair, and thereby remove it. One drawback to using the type of chemicals described in Mathews is that these chemicals may leave a tacky residue on the skin.

### SUMMARY OF THE INVENTION

The device and method for use of the device includes a substantially planar backing material having a surface, layer of a suitably depilatory adhesive adheres uniformly to the surface and is configured to adhere to and remove bodily hair and foreign debris. The practice of the method of applying the device to skin proximate to the surgical site is configured to entrap the hair and debris proximate to the site and to sever the hair and remove the debris upon stripping the backing material away from the skin, thereby reducing the time necessary to prepare a skin site for a surgery; preventing loss of the hair into the sterile environment; and protecting the epidermis of the skin from damage prior to the surgical preparation.

The device is additionally advantageous in that the method of use is intuitive and removing hair in a sterile, pre-packaged, ready-to-apply device that can be used by personnel with no training. The removal of the hair occurs at the ambient temperature of an operatory and without a further step of heating a depilatory wax. After stripping the backing material from the skin, the device is suitably folded into a containing membrane which itself is then sealed to form a closed container preventing hair and foreign debris from contaminating the sterile environment of the operatory.

Advantageously, in one embodiment, the containing membrane is suitably selected to be of sufficient size to accommodate the hand of the individual using the device. In the embodiment, the hand may be inserted into the membrane in order to position the backing material over the skin. The backing material is placed in adhesive engagement with the hair and foreign debris on the epidermis of the skin. The backing material is stripped from the skin, taking with it the hair and foreign debris. The containing membrane is uninverted thereby containing the backing material, the adhesive layer, and the foreign debris and hair. The containing membrane is sealed, thereby containing the hair and foreign debris outside of contaminating communication with the sterile environment of the operatory.

In a non-limiting embodiment, a device to remove potential contaminants from a region around a surgical site includes a first flexible sheet; a second flexible sheet coupled to the first flexible sheet to define a pouch; and an adhesive layer adjoined to an exposed side of one of the first or the second flexible sheets, wherein the pouch is invertible to bring the previously exposed sides of the flexible sheets into a face-to-face arrangement.

In another non-limiting embodiment, an apparatus to remove potential contaminants from a region around a surgical site includes a first flexible sheet; a second flexible sheet coupled to the first flexible sheet to define a pouch; an adhesive layer adjoined to an exposed side of one of the first or the second flexible sheets; and an engagement mechanism coupled to at least one of the first or second flexible sheets to selectively maneuver the adhesive layer of the pouch to pick up the potential contaminants from the region around the surgical site, wherein the pouch is invertible to bring the previously exposed sides of the flexible sheets into a face-to-face arrangement.

In yet another non-limiting embodiment, a method for removing potential contaminants from an area around a surgical site includes the steps of (1) moving an adhesive layer adjoined to one of a first sheet or a second sheet defining a pouch into contact with at least a portion of the area around the surgical site; (2) bonding the adhesive layer to at least some of the potential contaminants around the surgical site; and (3) urging the pouch away from the surgical site to contemporaneously remove the potential contaminants that came into contact with the adhesive layer.

In still yet another non-limiting embodiment, a method of manufacturing a device for removing potential contaminants from an area around a surgical site includes coupling a first sheet to a second sheet to define a pouch; and adjoining an adhesive layer to one of the exposed sides of the first sheet or the second sheet; wherein the pouch is sufficiently flexible to be inverted after the potential contaminants have been removed from the area around the surgical site.

As will be readily appreciated from the foregoing summary, the teaching herein provides a method and apparatus for entrapping and removing hair and debris proximate to a surgical site obviating the need for severing the hair by shaving.

### BRIEF DESCRIPTION OF THE DRAWINGS

Preferred and alternative embodiments of the present invention are described in detail below with reference to the following drawings.

FIG. 1 is an isometric view of a device for removing potential contaminants from a region around a surgical site according to one illustrated embodiment.

FIG. 2 is a plan view of the device of FIG. 1 showing an adhesive layer adjoined to the backing material of the device.

FIG. 3 is a cross-sectional view of the device of FIG. 1 taken along line 3-3 of FIG. 2.

### DETAILED DESCRIPTION EMBODIMENTS OF THE INVENTION

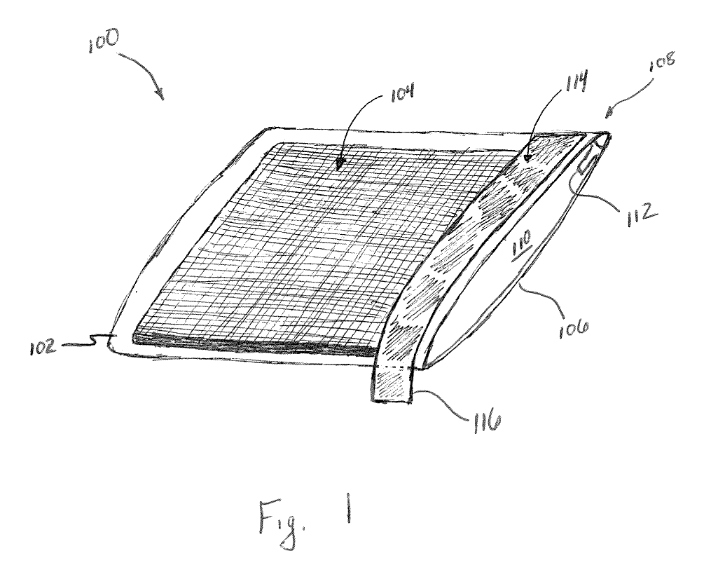

In an embodiment as depicted in FIG. 1, the device 100 comprises adhesive layer 104a flexible sheet or backing material 102 with an adhesive layer 104 adjoined thereto. The adhesive layer 104 could be composed of organic or synthetic compounds suitably configured to adhere to bodily hair or other debris that could contaminate a surgical site if not removed therefrom. When the backing material 102 is removed from the surgical site, the adhesive layer 104, having bonded to the body hair or other debris, pulls the hairs from the follicles while leaving the epidermis intact according to one embodiment. For the purposes of clarity and brevity in this description, body hair, debris, and other particulates that can be bonded to the adhesive layer 104 and subsequently removed from around a surgical site will be generally referred to as potential contaminants since one objective of removing the hair, debris, and other particulates is to prevent contamination of the surgical site.

The adhesive layer 104 exhibits a maximum bonding effectiveness at a predetermined temperature, which may be an average ambient temperature of an operating room, for example. Advantageously, the adhesive layer 104 does not have to be heated or cooled to be effective in adhering to the potential contaminants present on a patient's skin before or during a surgical procedure.

The backing material 102 includes any paper, non-woven felt, semi-porous cloth, or synthetic polymer film that is suitably selected to assure uniform and strong adhesion to the adhesive layer 104 thereby preventing the adhesive layer 104 from being pulled away from the backing material 102 in the course of potential contaminant removal. The backing material 102 is sufficiently flexible to be easily manipulated and conformable to various contours, curves, crevices, etc. of a patient's body. As a non-limiting example, the backing material 102 is made from muslin cloth.

In an embodiment and by way of example, the backing material 102 is coupled to a second flexible sheet 106, where the backing material 102 and the second flexible sheet 106 define a pouch 108 that includes a pocket, void, or opening 110. The second flexible sheet 106 is selected to exhibit suitable strength and tear-resistance to provide sufficient purchase on the backing material 102 during contaminant removal. In an embodiment, the pocket 110 in the pouch 108 is sized to receive at least a portion of a hand or an implement. In another embodiment, the second flexible sheet 106 also includes an adhesive layer (not shown) to allow the both sides of the pouch 108 to be employed for contaminant removal.

The pouch 108 includes the backing material 102, the second flexible sheet 106, and one or more adhesive layers 104 adjoined to at least one of the backing material 102 and/or the second flexible sheet 106 is invertible to define an inside-out pouch. After the pouch 108 has been inverted, the previously exposed surfaces of the pouch 108 are no longer exposed, but instead are arranged in a face-to-face relationship on the inside of the inverted pouch. The inverted pouch contains and captures the adhesive layer 104 with any entrapped contaminants. The previously exposed surfaces of the backing layer 102 and the second flexible sheet 106 are covered with a suitable material, such as a low density polyethylene polymer (LDPE) film, or other covering, coating, or protective layer. After inversion of the pouch 108, the film, which now forms the exterior of the inverted pouch, operates as a containment or storage baggie for the adhesive layer 104 and any attached contaminants.

The bonding agent of the adhesive layer 104 can be selected from a variety of bonding agents that are effective at bonding with hair and overcoming the coupling strength between the hair and the hair follicle. By way example, the bonding agent may be a polymer-hydrocarbon resin with a tackifier and a softener, a long-chain carbohydrate sugar with an alcohol such as glycerin, or several synthetic hydrocarbon polymers, where these bonding agents have a viscosity and bonding effectiveness sufficient to serve as the adhesive layer 104.

In yet another non-limiting embodiment, the adhesive layer 104 is optionally covered by a protective film (not shown) in opposing relationship to the backing material 102 relative to the adhesive layer 104. The protective film (not shown) may be, but is not limited to an acetate film, cellulose film, or waxed-paper. The protective film can be temporarily placed and retained on adhesive layer 104 to prevent unwanted airborne or other particulates from adhering to the adhesive layer 104 before it is used for its intended function in the surgical room. The protective layer can be easily removed from the adhesive layer 104 and does not diminish the adhesive properties of the adhesive layer 104.

When the device is used to remove contaminants from around a surgical site before or during a surgical procedure, the adhesive layer 104 is pressed against the patient's skin proximate to an incision site. A hand or implement received inside the pocket 110 of the pouch 108 urges the adhesive layer 104 into engagement with the skin and then away from the skin, thereby bonding with and removing hair, debris, and other contaminants.

The pouch may then be inverted by pulling the inner surfaces of the backing material 102 and the second flexible sheet 106 to form an inverted pouch. As previously mentioned, the inverted pouch now forms a container or storage device for the adhesive layer 104 and any contaminants bonded thereto. The storage device (i.e., inverted pouch) may include any of a number of commercially available sealable closure devices 112, such as a Zip-Loc® closure manufactured by S. C. Johnson Home Storage, Inc., or a peel-and-stick adhesive strip 114. In one embodiment, the peel-and-stick adhesive strip includes a tab 116 attached to the strip 114 for easier manipulation of the strip. Upon inversion and sealing, the adhesive layer 104 and contaminants are sealed within the inverted pouch for disposal.

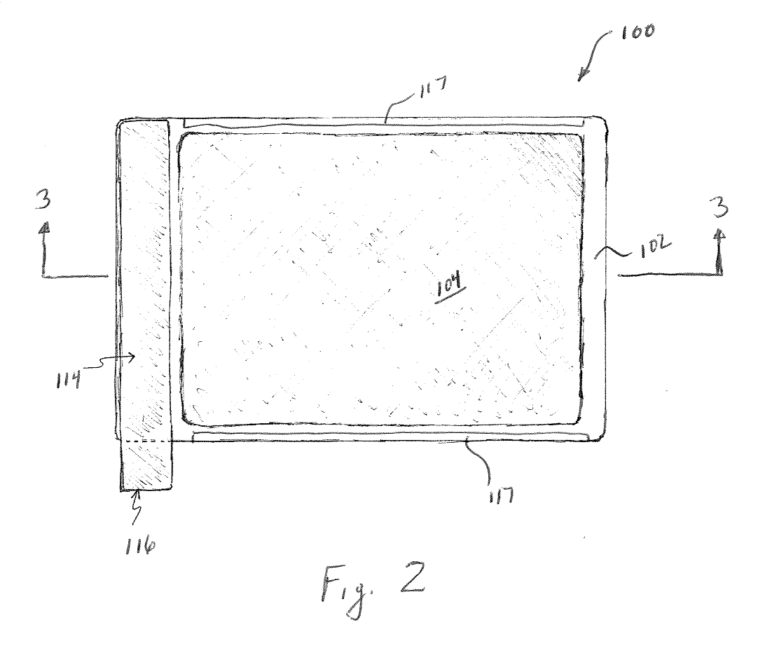

FIG. 2 shows another embodiment of the device 100 depicting an arrangement of the backing material 102 along with the second flexible sheet 106, the adhesive layer 104, the protective strip 114, and a sealing adhesive tab 116. In addition, the depicted device 100, as a non-limiting embodiment, provides edge members 117 for engaging an implement (not shown), which may be a tool configured to grip the edge members 117 and maneuver the pouch into contact with the patient's skin. The tool may be used in conjunction with another implement, such as a portion of a hand, placed inside the pouch to selectively press on the skin.

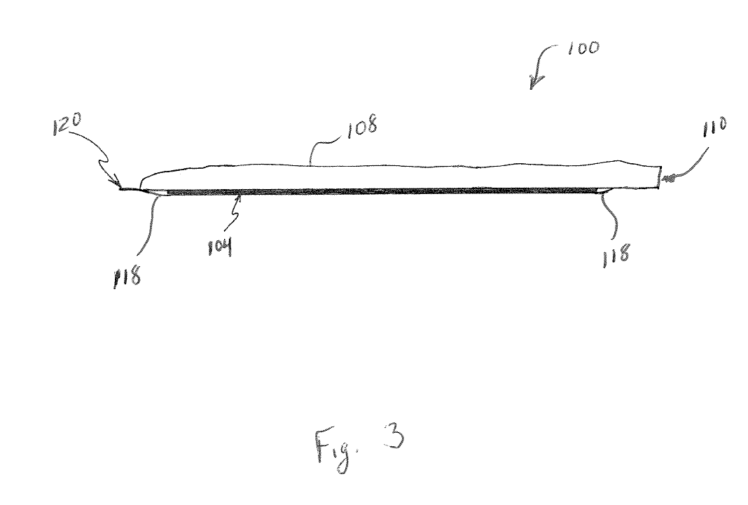

FIG. 3 shows another embodiment of the device 100 in which the adhesive layer 104 is suitably adhered to the pouch 108. The device 100 further includes a protective film 118 with an optional, grippable tab 120 to facilitate removal of the protective film 118 from the adhesive layer 104. The pouch 108 is configured to receive at least a portion of a hand in an opening 110 of the pouch 108. The flexibility of the pouch 108 permits it to be urged onto and conformed with the patient's skin as well as permit the pouch 108 to be inverted after the contaminants are removed from the skin.

An advantage of at least one embodiment of the described device is that the device is easily invertible and sealable after use, which prevents the removed contaminants from potentially re-entering the sterile operating or surgical environment. Yet another advantage is that the device not only removes hair and debris, but may further remove some of the patient's epidermal cells, thus exposing a more sterile sub-layer of the skin.

The device 100 and procedures for using the device for entrapping and removing contaminants proximate a surgical site obviates the need to shave a patient's body hair with a razor, which in turn reduces the time to prep the surgical area and substantially reduces, if not eliminates, the potential for waterborne contaminants to remain on the skin after shaving. The device 100 may be configured in a multitude of sizes and shapes to fit various sized implements, such as various sized hands. While various embodiments of the invention have been illustrated and described, as noted above, many changes can be made without departing from the spirit and scope of the invention. Accordingly, the scope of the invention is not limited by the disclosure of the embodiments, but instead is determined by the claims below.

The embodiments of the invention in which an exclusive property or privilege is claimed are defined as follows:

1. A device to remove potential contaminants from a region around a surgical site, the device comprising:
a first flexible sheet;
a second flexible sheet coupled to the first flexible sheet to define a pouch; and
an adhesive layer adjoined to an exposed side of one of the first or the second flexible sheets,
wherein the pouch is invertible to bring the previously exposed sides of the flexible sheets into a face-to-face arrangement.

2. The device of claim 1, wherein the pouch is sized to receive at least a portion of a hand.

3. The device of claim 1, wherein the pouch is sized to receive an implement.

4. The device of claim 1, wherein the adhesive layer includes a bonding effectiveness that is sufficient to remove a significant number of the potential contaminants from the region around the surgical site.

5. The device of claim 1, wherein the bonding effectiveness occurs within approximately a first temperature range.

6. The device of claim 1, further comprising:
a first edge member coupled to a first closed edge of the pouch; and
a second edge member coupled to a second closed edge of the pouch, the first closed edge located opposite the second closed edge.

7. The device of claim 1, wherein the edge members are engageable by an implement external to the pouch.

8. The device of claim 7, wherein the implement is a tool.

9. An apparatus to remove potential contaminants from a region around a surgical site, the device comprising:
a first flexible sheet;
a second flexible sheet coupled to the first flexible sheet to define a pouch;
an adhesive layer adjoined to an exposed side of one of the first or the second flexible sheets; and
edge members coupled to at least one of the first or second flexible sheets to allow the pouch to be engaged by an implement and selectively maneuvered where the adhesive layer bonds with the potential contaminants from the region around the surgical site,
wherein the pouch is invertible to bring the previously exposed sides of the flexible sheets into a face-to-face arrangement.

10. The apparatus of claim 9, wherein the edge members are removeable from the pouch before the pouch is inverted.

11. The apparatus of claim 9, wherein the edge members comprise a stitched, fabric perimeter located on a first and an opposing edge of the pouch.

12. A method for removing potential contaminants from an area around a surgical site, the method comprising:
moving an adhesive layer adjoined to one of a first sheet or a second sheet defining a pouch into contact with at least a portion of the area around the surgical site;
bonding the adhesive layer to at least some of the potential contaminants around the surgical site; and
urging the pouch away from the surgical site to contemporaneously remove the potential contaminants that came into contact with the adhesive layer.

13. The method of claim 12, further comprising:
inverting the pouch to maintain the potential contaminants in a pocket formed by the inverted pouch.

14. The method of claim 12, further comprising:
sealing the inverted pouch to prevent the potential contaminants from being discharged into a surgical environment.

15. The method of claim 12, wherein urging the pouch away from the surgical site includes grasping one of the first sheet or the second sheet of the pouch.

16. A method of manufacturing a device for removing potential contaminants from an area around a surgical site, the method comprising:
coupling a first sheet to a second sheet to define a pouch; and
adjoining an adhesive layer to one of the exposed sides of the first sheet or the second sheet;
wherein the pouch is sufficiently flexible to be inverted after the potential contaminants have been removed from the area around the surgical site.

17. The method of claim 16, further comprising:
covering the adhesive layer with a removable sheet to protect the adhesive layer from degradation.

18. The method of claim 16, wherein coupling the first sheet to the second sheet to define the pouch includes forming edge members along opposing sides of the pouch.
